(19) United States
(12) Patent Application Publication
(10) Pub. No.: US 2010/0075360 A1
(43) Pub. Date: Mar. 25, 2010

(54) UV BASED CELL VIABILITY AS AN INDICATOR OF SUN PROTECTION FACTOR AND METHODS OF MEASUREMENT THEREOF

(76) Inventors: Muhammed Majeed, Piscataway, NJ (US); Susmitha Anand, Bangalore (IN); Geetha Kanhangad Gangadharan, Bangalore (IN); Subbalakshmi Prakash, Piscataway, NJ (US)

Correspondence Address: SABINSA CORPORATION, 70 ETHEL ROAD WEST, UNIT 6, PISCATAWAY, NJ, 08854, US

(21) Appl. No.: 12/234,804

(22) Filed: Sep. 22, 2008

(51) Int. Cl. C12Q 1/02

(52) U.S. Cl. 435/29

(57) ABSTRACT

Disclosed is a simple method of determining SPF based on the staining pattern of viable cells like mouse fibroblasts and human keratinocytes after exposure to ultraviolet radiations. The method provides results comparable to the standard methods of SPF measurement using human subjects.

### BACKGROUND OF THE INVENTION

1. Field of the Invention

The present invention relates to methods of measuring “Sun Protection Factor” (SPF). More specifically, the present invention discloses a simple method of determining SPF based on the staining pattern of viable cells like mouse fibroblasts and human keratinocytes after exposure to ultraviolet radiations. The method provides results comparable to the standard methods of SPF measurement using human subjects.

2. Description of Prior Art

Methods to Determine Sun Protection Factor as Available in Prior Art Include:

WO/2005/103659 to Ferrero, Louis on Mar. 11, 2005 wherein a method for determining an integral sun protection factor (ò SPF or iSPF) which encompasses both UVA and UVB radiation and can be used for classifying cosmetic and dermatological sunscreens has been disclosed.

EP1291640 to Wendel, Dr. Volker on May 9, 2001 discusses an in-vitro test method to measure the UVA protection performance (UVAPF) of sun care products by measuring the in-vitro absorbance data, normalising the spectrum to the in-vivo SPF and calculating the UVA protection performance by the following formula: EMI12.1 Where E(lambda)=irradiance at wavelength lambda of the light spectrum used C=constant factor for the adjustment of the spectrum to the in-vivo measurement S(lambda)=effectiveness of a biological UVA endpoint at wavelength lambda A(lambda)=absorbance.

Evaluation of the efficiency of sun care products has for a long time been assessed through the in vivo sun protection factor (SPF) test, which is performed on human volunteers. Introduced across several decades, the SPF method is based on a human end-point well-known in biology: the minimal erythemal dose (MED), mainly due to the ultraviolet B (UVB) part of the sun spectrum radiations. The increase in level of protection, according to increasing safety standards for the consumer, leads to higher SPF values—often over 30—which are more difficult to measure. A high SPF normally leads to a greater uncertainty in the final in vivo result. The in vivo method is time-consuming, impossible to run all year round and expensive—not to mention the ethical problems of testing on humans. For economical, practical and ethical reasons, a reliable in vitro measurement of the SPF which seemed particularly useful as a supplement to the in vivo SPF was so far based on the physical and optical determination of the reduction of the energy in the UV range, through a film of product which has previously been spread on an adequate substrate.

Accordingly it is the principle object of the present invention to develop a simple in-vitro method of determining SPF based on the staining pattern of viable cells like mouse fibroblasts and human keratinocytes after exposure to ultraviolet radiations.

It is another object of the present invention to develop a simple in-vitro method of determining SPF that provides results similar to clinical determinations without involving animal or human volunteers and which takes lesser time to perform.

The present invention fulfills the aforesaid objectives and provides further related advantages.

### SUMMARY OF THE INVENTION

The present invention is a simple method of determining SPF based on the staining pattern of viable cells like mouse fibroblasts and human keratinocytes after exposure to ultraviolet radiations. After morphological analysis of the viable cells, the stain was dissolved and its optical density was determined using a microtiter plate assay system. The SPF of well known sun blockers like Octyl methoxy cinnamate and Galanga extract from Kaempferia galanga root was determined by the in vitro methodology and compared to that of the clinically determined SPF. Similarly, the SPF of various sunscreen formulations was determined and compared to that determined clinically. The SPF determined by both the methodologies was observed to be similar and hence the in vitro methodology has major advantages.
(i) The assay is easy to perform and specific with respect to different cell lines.
(ii) The assay does not involve animals or human volunteers,
(iii) The assay takes lesser time period of study;
(iv) The assay provides results similar to clinical SPF study.
(v) The assay is adaptable to unformulated actives in either liquid or solid form.
(vi) The assay is capable of detecting phototoxic compounds which can be eliminated.
(vii) Since the quantification is based on microtiter plate reading, the method can be recommended for the screening of large number of samples.

Other features and advantages of the present invention will become apparent from the following more detailed description, taken in conjunction with the accompanying drawings, which illustrate, by way of example, the principle of the invention.

### BRIEF DESCRIPTION OF THE DRAWINGS

FIG. 1 shows the effect of UV on Swiss 3T3 fibroblast cells at varyng dosages as determined by Sulphorhodamine B (SRB) staining, The neutral red (NR) uptake assay and 3-(4,5-dimethylthiazol-2-yl)-2-5-diphenyltetrazolium bromide (MTT) assay.

FIG. 2 shows the effect of UV on HaCAT human keratinocyte cells at varying dosages as determined by Sulphorhodamine B (SRB) staining, The neutral red (NR) uptake assay and 3-(4,5-dimethylthiazol-2-yl)-2-5-diphenyltetrazolium bromide (MTT) assay.

FIG. 3 shows the effect of UV on unprotected Swiss 3T3 fibroblasts in comparison to that of 1.25% OMC treated cells.

FIG. 4 shows the effect of UV on unprotected Swiss 3T3 fibroblasts in comparison to that of 7% OMC treated cells.

FIG. 5 shows the effect of UV on unprotected Swiss 3T3 fibroblasts in comparison to that of 0.25% Galanga extract treated cells.

FIG. 6 shows the effect of UV on unprotected Swiss 3T3 fibroblasts in comparison to that of cells treated with a sunscreen formulation CS/NEE/15.

### DESCRIPTION OF THE MOST PREFERRED EMBODIMENT

In the most preferred embodiment the present invention relates to

An in-vitro method for determining sun protection factor, said method comprising the steps of:
(i) Growing suitable cell cultures for 24 hours to form cell monolayers;
(ii) Exposing the cell monolayers of step (i) to UV irradiation dosages in presence and absence of sample whose Sun protection factor needs to be measured;
(iii) Staining the UV irradiated monolayers of step (ii) 48 hours after UV irradiation; and
(iv) Quantitating cell death occurring due to UV exposure in the presence and absence of the sample of step (ii) for the determination of Sun Protection factor. More preferably, the cell monolayer is mouse fibroblasts or human keratinocytes. In another preferred embodiment the staining method may be one selected from the group consisting of Sulphorhodamine B (SRB) staining method, the neutral red (NR) uptake assay or the 3-(4,5-dimethylthiazol-2-yl)-2-5-diphenyltetrazolium bromide (MTT) assay

The most preferred embodiment of the present invention is illustrated through the examples sighted herein below.

### EXAMPLE I

### Materials and Methods

Cell Cultures

Swiss 3T3 Mouse fibroblast cells and HaCAT Human keratinocytes were obtained from ATCC and cultured in DMEM medium supplemented with 10% FBS. The confluent cultures are harvested by trypsinization and expanded during two more passages before they were used for the experiments. Medium and other culture components were renewed after 48-72 h. All cell cultures were maintained in a humidified atmosphere at 37° C. in 95% air and 5% CO₂.

UV Source

UV lamp of 14.7 W, 0.3 A, 55V with an intensity of 33.3 μW/cm² from Sankyo Denki Co., Ltd.

Cell Staining Techniques

Sulphorhodamine B (SRB) staining: SRB was purchased from Sigma. The dye was dissolved in 1% acetic acid at a concentration of 400 mg/100 ml. SRB dye is stable for months at RT. The SRB assay is used for cell density determination, based on the measurement of cellular protein content. The method described here has been optimized for the toxicity screening of compounds to adherent cells in a 96-well format. After UV exposure and consequent incubation for 72 hrs, cell monolayers are fixed with 10% (w/v) trichloroacetic acid and stained for 30 min, after which the excess dye is removed by washing repeatedly with 1% (v/v) acetic acid. The protein-bound dye is dissolved in 10 mM Tris base solution for OD determination at 492 nm using a microplate reader. The results are linear over a 20-fold range of cell numbers and the sensitivity is comparable to those of fluorometric methods. The method not only allows a large number of samples to be tested within a few days, but also requires only simple equipment and inexpensive reagents. The SRB assay is therefore an efficient and highly cost-effective method for screening.

The neutral red (NR) uptake assay: The NR cytotoxicity assay procedure is a cell survival/viability chemosensitivity assay, based on the ability of viable cells to incorporate and bind neutral red, a supravital dye. NR is a weak cationic dye that readily penetrates cell membranes by non-ionic diffusion, accumulating intracellularly in lysosomes, where it binds with anionic sites in the lysosomal matrix. Alterations of the cell surface or the sensitive lysosomal membrane lead to lysosomal fragility and other changes that gradually become irreversible. Such changes brought about by UV exposure result in a decreased uptake and binding of NR. It is thus possible to distinguish between viable, damaged, or dead cells, which are the basis of this assay. The dye taken up by the cells is subsequently extracted and measured. The dye was dissolved in distilled water at a concentration of 0.5 mg/100 μl. 80 μl of this stock solution was dissolved in 15 ml of pre warmed DMEM medium. The developer solution consists of 25 ml of water, 24.5 ml of ethanol and 0.5 ml of glacial acetic acid. The NRU dye and the developer solutions are to be made fresh before use. After UV exposure and consequent incubation for 72 hrs, the culture medium is replaced with NRU dye and incubated for 3 hrs at 37° C., after which the dye is washed off thrice with PBS. The dye is dissolved in 100 μl of developer solution and incubated at RT for 20 min. The OD was determined at 492 nm using a microplate reader.

3-(4,5-dimethylthiazol-2-yl)-2-5-diphenyltetrazolium bromide (MTD assay: The MTT cytotoxicity assay measures the metabolically inactive cells. Since the tetrazolium salts are reduced to formazen by the metabolically active cells, this assay detects viable cells exclusively. Any changes brought about by UV exposure results in a decreased uptake and binding of MTT as the cells become metabolically inactive. The dye taken up by the cells is subsequently extracted and measured. The dye was dissolved in PBS at a concentration of 5 mg/ml. After UV exposure and consequent incubation for 72 hrs, 20 μl of MTT dye was added per well and incubated for 3 hrs at 37° C., after which the dye is washed off with PBS. The dye is dissolved in 200 μl of DMSO. The OD was determined at 492 nm using a microplate reader.

Cell culture and UV exposure: The cells were plated in a 96 well micropipette at a seeding density of 3000 cells per well for fibroblasts and 10000 cells per well for keratinocytes. The 24 hr monolayers of cells were exposed to UV B source at a distance of 30 cm for varying time intervals to attain varying UV dosages. After each exposure dose, the exposed region of the plate was covered by aluminum foil. After exposure, the cells were incubated in a CO₂ incubator for 48 hrs and developed by the varying staining techniques to analyze the cell viability.

Results

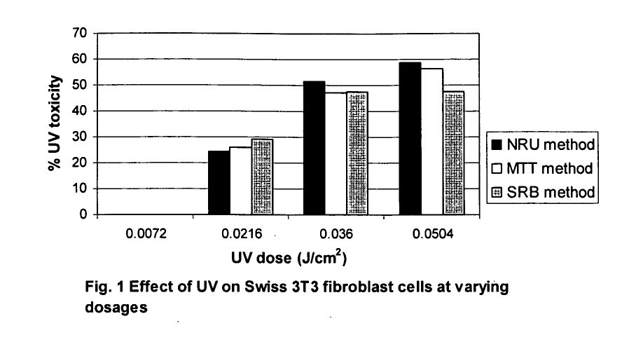

As observed in Table 1 and FIG. 1, cell death was initiated at a dosage of 0.0216 J/cm² in Swiss 3T3 fibroblast cells as determined by all the three cell staining techniques, NRU, MTT and SRB techniques. Hence, the minimal cytotoxic dose (MCDu) for unprotected cells is 0.0216 J/cm².

TABLE 1

| UV | | | |
|---|---|---|---|
| dosage | % UV toxicity in Swiss 3T3 fibroblast cells | | |
| (J/cm²) | NRU method | MTT method | SRB method |
| 0.0072 | 0 | 0 | 0 |
| 0.0216 | 24.3 | 26 | 29.3 |
| 0.036 | 51.4 | 47.2 | 47.8 |
| 0.0504 | 58.7 | 56.35 | 47.8 |

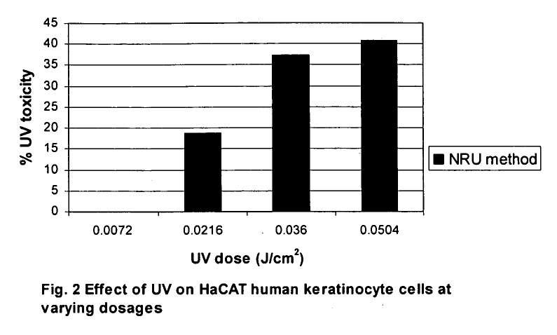

Similarly, in HaCAT human keratinocyte cells, cell death was initiated at a dosage of 0.0216 J/cm² as observed in Table 2 and FIG. 2. Hence, the minimal cytotoxic dose (MCDu) for unprotected cells is 0.0216 J/cm².

TABLE 2

| | % UV toxicity in HaCAT human |
|---|---|
| UV dosage (J/cm²) | keratinocyte cells |
| 0.0072 | 0 |
| 0.0216 | 18.8 |
| 0.036 | 37.2 |
| 0.0504 | 40.8 |

Determination of SPF of Unformulated Actives:

Determination of Percentage of UV Protection by OMC in Swiss 3T3 Fibroblast Cells:

Swiss 3T3 fibroblasts were plated in a 96 well micropipette at a seeding density of 3000 cells per well. Varying concentrations of OMC in the cell culture medium were added to the 24 hr monolayers of cells and then exposed to UV B source at a distance of 30 cm for 5 minutes. The unexposed region of the plate was covered by aluminum foil. After exposure, the cells were incubated in a CO₂ incubator for 48 hrs and developed by the NRU staining techniques to analyze the cell viability. The percentage of UV protection was calculated with respect to unexposed cells.

TABLE 3

| Conc. Of OMC (%) | % UV protection |
|---|---|
| 0.15 | 56 |
| 0.31 | 71 |
| 0.62 | 77 |
| 1.25 | 90 |

Determination of SPF of 1.25% OMC in Swiss 3T3 Fibroblast Cells:

Swiss 3T3 fibroblasts were plated in a 96 well micropipette at a seeding density of 3000 cells per well. The concentration of OMC giving maximum UV protection, that is, 1.25% concentration of OMC in the cell culture medium was added to the 24 hr monolayers of cells and then exposed to UV B source at a distance of 30 cm for varying time intervals of 1, 3, 5, 7, 10, 20 and 30 minutes for increasing UV dosages. A control plate without OMC treatment was also exposed under similar conditions. After exposure, the cells were incubated in a CO₂ incubator for 48 hrs and developed by the NRU staining technique to analyze the cell viability. The SPF of 1.25% OMC was determined with respect to the untreated cells.

TABLE 4

| | |
|---|---|
| Control plate | 1.25% OMC treated plate |

| UV dosage | % UV | UV dosage | |
|---|---|---|---|
| (J/cm²) | toxicity | (J/cm²) | % UV toxicity |
| 0.0072 | 0 | 0.0072 | 0 |
| 0.0216 | 20.5 | 0.0216 | 0 |
| 0.036 | 44.4 | 0.036 | 0 |
| 0.0504 | 57 | 0.0504 | 0 |
| 0.072 | 100 | 0.072 | 0 |
| 0.144 | 100 | 0.144 | 27.36 |
| 0.216 | 100 | 0.216 | 39.62 |

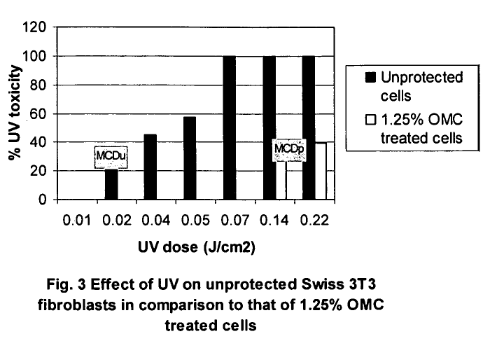

As observed in Table 4 & FIG. 3, the minimal cytotoxic dose (MCDu) for unprotected cells is 0.0216 J/cm² and the minimal cytotoxic dose (MCDp) for protected cells is 0.144 J/cm²

SPF=MCDp/MCDu

SPF of 1.25% OMC=0.144/0.0216=6.66

Determination of SPF of 7% OMC in Swiss 3T3 Fibroblast Cells:

Similarly, SPF of 7% OMC was also determined as 7% OMC is generally used in most of the sunscreen formulations.

TABLE 5

| | |
|---|---|
| Control plate | 7% OMC treated plate |

| UV dosage | % UV | UV dosage | |
|---|---|---|---|
| (J/cm²) | toxicity | (J/cm²) | % UV toxicity |
| 0.0072 | 0 | 0.0072 | 0 |
| 0.0216 | 20.5 | 0.0216 | 0 |
| 0.036 | 44.4 | 0.036 | 0 |
| 0.0504 | 57 | 0.0504 | 0 |
| 0.072 | 100 | 0.072 | 0 |
| 0.144 | 100 | 0.144 | 0 |
| 0.18 | 100 | 0.18 | 0 |
| 0.432 | 100 | 0.432 | 0 |
| 0.54 | 100 | 0.54 | 7 |
| 0.648 | 100 | 0.648 | 9 |

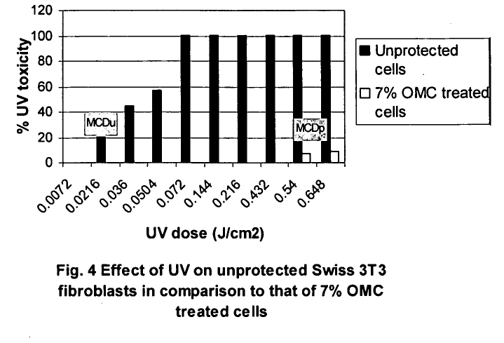

As observed in Table 5 & FIG. 4, the minimal cytotoxic dose (MCDu) for unprotected cells is 0.0216 J/cm² and the minimal cytotoxic dose (MCDp) for protected cells is 0.54 J/cm²

SPF of 7% OMC=0.54/0.0216=25

Determination of Percentage of UV Protection by Galanga Extract in Swiss 3T3 Fibroblast Cells:

Kaempferia galanga root extract is valued traditionally for its skin protectant action. One patented application of Kaempferia galanga pertains to its action against ultraviolet rays and function as a ‘booster’ that augments the activity of conventional sunscreens. Kaempferia galanga rhizome contains about 1.5 to 2% essential oil, whose main components are ethyl cinnamate (25%), ethyl p-methoxycinnamate (30%) and p-methoxycinnamic acid. Kaempferia galanga is a good natural source of a biologically active ester compound ethyl p-methoxycinnamate.

Percentage of UV protection and SPF of Galanga extract was studied similarly.

TABLE 6

| Conc. Of Galanga extract | |
|---|---|
| (%) | % UV protection |
| 0.031 | 73.8 |
| 0.062 | 82.7 |
| 0.125 | 100 |
| 0.25 | 100 |

Determination of SPF of 0.25% Galanga Extract in Swiss 3T3 Fibroblast Cells:

TABLE 7

| | |
|---|---|
| | 0.25% Galanga extract treated |
| Control plate | plate |

| UV dosage | % UV | UV dosage | |
|---|---|---|---|
| (J/cm²) | toxicity | (J/cm²) | % UV toxicity |
| 0.0072 | 0 | 0.0072 | 0 |
| 0.0216 | 15 | 0.0216 | 0 |
| 0.036 | 36 | 0.036 | 0 |
| 0.0504 | 48.4 | 0.0504 | 0 |
| 0.072 | 100 | 0.072 | 0 |
| 0.144 | 100 | 0.144 | 0 |
| 0.216 | 100 | 0.216 | 11.43 |

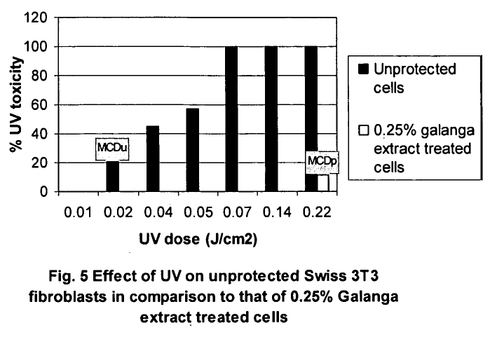

As observed in Table 7 & FIG. 5, the minimal cytotoxic dose (MCDu) for unprotected cells is 0.0216 J/cm² and the minimal cytotoxic dose (MCDp) for protected cells is 0.216 J/cm²

SPF of 0.25% Galanga extract=0.216/0.0216=10

Determination of SPF of Formulations:

Swiss 3T3 fibroblasts were plated in a 96 well micropipette at a seeding density of 3000 cells per well. The 24 hr monolayers of cells were covered by a polystyrene lid and then exposed to UV B source at a distance of 30 cm for varying time intervals for increasing UV dosages. Similarly another 96 well plate containing 24 hr monolayers was covered by a polystyrene lid and a sunscreen formulation CS/NEE/115 was applied over the lid at a dose of 20 mg over each well uniformly spread in an area of 0.4 cm². The sunscreen formulation had highly effective actives in the following concentrations:

### OMC—7%

### Tinosorb—5%

### Boswellin—1%

### Arbutin—0.5%

### Licorice—0.1%

Coriander seed oil—0.3%

After exposure, the cells were incubated in a CO₂ incubator for 48 hrs and developed by the NRU staining technique to analyze the cell viability. The SPF of the sunscreen formulation was determined with respect to the untreated cells.

TABLE 8

| | |
|---|---|
| Control plate | CS/NEE/115 treated plate |

| UV dosage | | UV dosage | % UV |
|---|---|---|---|
| (J/cm²) | % UV toxicity | (J/cm²) | toxicity |
| 0.0072 | 0 | 0.0072 | 0 |
| 0.0216 | 0 | 0.0216 | 0 |
| 0.036 | 0 | 0.036 | 0 |
| 0.0504 | 0 | 0.0504 | 0 |
| 0.072 | 13.7931 | 0.072 | 0 |
| 0.144 | 37.64368 | 0.144 | 0 |
| 0.18 | 49.13793 | 0.18 | 0 |
| 0.216 | 50.86207 | 0.216 | 0 |
| 0.432 | 60 | 0.432 | 0 |
| 0.54 | 100 | 0.54 | 0 |
| 0.864 | 100 | 0.864 | 0 |
| 1.296 | 100 | 1.296 | 16.36 |
| 1.512 | 100 | 1.512 | 35.63 |

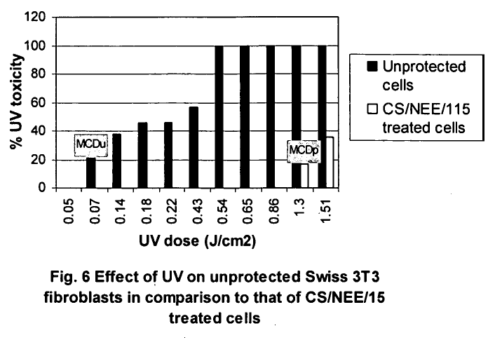

As observed in Table 8 & FIG. 6, the minimal cytotoxic dose (MCDu) for unprotected cells is 0.072 J/cm² and the minimal cytotoxic dose (MCDp) for protected cells is 1.296 J/cm²

SPF of CS/NEE/115 sunscreen formulation=1.296/0.072=18

Comparison of the in vitro cell based SPF determined with that determined by the clinical study conducted for CS/NEE/115 sunscreen formulation:

The clinical study was conducted for CS/NEE/115 sunscreen formulation for SPF determination by Clinical Research Laboratories, Inc. New Jersey. As per the final study report, study no. CRL172607, the SPF of the formulation was found to be 18.

Discussion

The in vitro method presented here has major advantages such as non involvement of animals or human volunteers, lesser time period of study and similarity to clinical SPF study. Moreover, unlike in the clinical study, SPF can be determined for unformulated actives also.

The NRU staining technique was observed to be more effective for determining the cell viability after UV exposure for UV protection and SPF calculations. The UV protection and SPF analysis was observed to be specific for different cell lines. The fibroblast cells were found to be more vulnerable as compared to the keratinocytes.

As shown in Tables 1 & 2, the UV toxicity was in the range of 24-58% for fibroblast cells where as the UV toxicity was in the range of 19-41% for keratinocyte cells after UV exposure.

As shown in Table 3, OMC showed a maximum UV protection of 90% at a concentration of 1.25%. The SPF of unformulated active, for example, OMC was thus determined at this concentration and was found to be about 7 as shown in Table 4. Similarly, Galanga extract showed 100% UV protection at 0.125 and 0.25% concentrations as shown in Table 5. The SPF was determined at 0.25% concentration for Galanga extract and was found to be 10 as shown in Table 6. Hence, Galanga extract was superior to OMC for SPF. However, a higher concentration of OMC will give a higher SPF value as is generally observed in clinical studies. The SPF value for 7% OMC was 25 as shown in Table 7.

As shown in Table 5, a sunscreen formulation with effective UV absorbers produced similar SPF result of 18 in the cell based in vitro method as compared to the clinical study conducted by Clinical Research laboratories, which also produced the similar results, SPF 18. Hence, the proposed in vitro cell based technique of determining SPF is far superior to the clinical technique involving human volunteers.

The method is adaptable for the determination of SPF through markers other than cell viability. Such markers may signify inflammation or free radical oxidative damage.

While the invention has been described with reference to a preferred embodiment, it is to be clearly understood by those skilled in the art that the invention is not limited thereto. Rather, the scope of the invention is to be interpreted only in conjunction with the appended claims.

We claim:

1. An in-vitro method for determining sun protection factor, said method comprising the steps of:
(i) Growing suitable cell cultures for 24 hours to form cell monolayers;
(ii) Exposing the cell monolayers of step (i) to UV irradiation dosages in presence and absence of sample whose Sun protection factor needs to be measured;
(iii) Staining the UV irradiated monolayers of step (ii) 48 hours after UV irradiation; and
(iv) Quantitating cell death occurring due to UV exposure in the presence and absence of the sample of step (ii) for the determination of Sun Protection factor.

2. The method according to claim 1, wherein the cell monolayer is mouse fibroblasts.

3. The method according to claim 1, wherein the cell monolayer is human keratinocytes.

4. The method according to claim 1, wherein the staining is done by the Sulphorhodamine B (SRB) staining method.

5. The method according to claim 1, wherein the staining is done by the neutral red (NR) uptake assay.

6. The method according to claim 1, wherein the staining is done by the 3-(4,5-dimethylthiazol-2-yl)-2-5-diphenyltetrazolium bromide (MTT) assay.
